(19) United States
(12) Patent Application Publication
(10) Pub. No.: US 2008/0045495 A1
(43) Pub. Date: Feb. 21, 2008

(54) Treatment of Patients with Cystic Disease by Alteration of Fibrocystin Proteolysis

(75) Inventors: Thomas R. Hiesberger, Dallas, TX (US); Peter Igarashi, Plano, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/829,768

(22) Filed: Jul. 27, 2007

Related U.S. Application Data
(60) Provisional application No. 60/820,573, filed on Jul. 27, 2006.

(51) Int. Cl. A61K 31/415; A61K 31/195; A61K 31/27; A61K 31/335; A61K 31/437; A61P 13/12; A61K 31/343; A61K 31/397; A61K 31/403

(52) U.S. Cl. 514/210.16; 514/299; 514/386; 514/396; 514/423; 514/450; 514/473; 514/506; 514/561

(57) ABSTRACT

The present invention includes methods and compositions for reducing cyst formation in a patient with polycystic kidney disease by contacting the kidney of the patient with an effective amount of a fibrocystin cleavage inhibitor sufficient to reduce cyst formation, wherein the fibrocystin cleavage inhibitor comprises at least one of a proteasome inhibitor, a calpain inhibitor, and a β-secretase inhibitor and reduces the degradation of the fibrocystin cleavage products.

### CROSS-REFERENCE TO RELATED APPLICATIONS

This application claims priority to U.S. Provisional Application Ser. No. 60/820,573, filed Jul. 27, 2006, the contents of which is incorporated by reference herein in its entirety.

### STATEMENT OF FEDERALLY FUNDED RESEARCH

This invention was made with U.S. Government support under Contract No. R01 DK-67565 awarded by the NIH. The government has certain rights in this invention.

### TECHNICAL FIELD OF THE INVENTION

The present invention relates in general to the field of polycystic kidney disease, and more particularly, to compositions and methods for the reduction of cyst formation caused by alterations of Fibrocystin proteolysis.

### BACKGROUND OF THE INVENTION

Without limiting the scope of the invention, its background is described in connection with polycystic kidney diseases and more specifically compositions and methods for the reduction of cyst formation caused by alterations of Fibrocystin proteolysis.

In U.S. Pat. No. 7,045,303, Wilson, et al., teach screening methods for compounds useful in the treatment of polycystic kidney disease. More particularly, a cell-based screening assay designed to identify agents that regulate the activity of the polycystic kidney disease proteins encoded by the PKD-1 and PKD-2 genes and that may be useful in the treatment of polycystic kidney disease is disclosed. The assays include contacting of genetically engineered cells expressing a mutant or truncated PKD gene product with a test agent and assaying for a decrease in the PKD mediated mutant phenotype including, e.g., interacting proteins. Interacting proteins include, for example, focal adhesion complex proteins such as FAK, paxillin, vinculin, talin and the like.

Yet another patent is U.S. Pat. No. 6,875,747, by Iversen, et al. to the use of antisense c-myc for treatment of polycystic kidney disease. Briefly, a method of treating polycystic kidney disease by administering an oligonucleotide antisense to c-myc is described. The antisense oligonucleotide is preferably a morpholino oligonucleotide.

Another patent is U.S. Pat. No. 5,972,882, to Gattone II, for the treatment of polycystic kidney disease using vasopressin V₂ receptor antagonists. This invention is directed to the novel treatment of ARPKD and ADPKD by administering a pharmacologically effective amount of a V₂ receptor antagonist. Orally active V₂ receptor antagonists such as OPC-31260, OPC-41061, SR121463A and VPA-985 are administered alone, or in combination to mammalian PKD subjects to reduce the cAMP generated by the increased expression of AVP-V₂ receptor, AQP2 and AQP3, thereby reducing and/or preventing cyst enlargement.

Yet another disclosure is found in United States Patent Application No. 20040024042, Breyer, Matthew for a COX2 inhibition in the prevention and treatment of autosomal dominant polycystic kidney disease. The present invention provides a new therapeutic approach for autosomal dominant polycystic kidney disease (ADPKD). Cyclooxygenase 2 (COX2) inhibitors are used, alone or in combination with other drugs, to prevent or limit early stage cyst formation.

### SUMMARY OF THE INVENTION

The present invention provides a method of reducing cyst formation in a patient with polycystic kidney disease by contacting the kidney of the patient with an effective amount of a fibrocystin cleavage inhibitor sufficient to reduce cyst formation, wherein the fibrocystin cleavage inhibitor includes at least one of a proteasome inhibitor, a calpain inhibitor, and a secretase inhibitor and reduces the degradation of the fibrocystin cleavage products.

The present invention also includes a composition for reducing cyst formation in a patient with polycystic kidney disease. The composition includes an effective amount of a fibrocystin cleavage inhibitor sufficient to reduce cyst formation in a patient, wherein the fibrocystin cleavage inhibitor includes at least one of a proteasome inhibitor, a calpain inhibitor, and a β-secretase inhibitor.

The present invention provides a method of reducing cyst formation in a patient with polycystic kidney disease by contacting the kidney of the patient with an effective amount of a fibrocystin cleavage inducer sufficient to reduce cyst formation, wherein the fibrocystin cleavage inducer comprises at least one agent that increases an intracellular Ca⁺⁺ concentration wherein the levels of fibrocystin cleavage products increases.

A method of reducing cyst formation in a patient with polycystic kidney disease by contacting the kidney of the patient with an effective amount of a fibrocystin cleavage inducer sufficient to reduce cyst formation, wherein the fibrocystin cleavage inducer includes at least one agent that increases the activity of Protein Kinase C is also provided by the present invention.

The present invention also provides a method of reducing cyst formation in a patient with polycystic kidney disease by contacting the kidney with one or more agents that induce fibrocystin cleavage selected from modulators of microtubule polarization, modulators of Actin polymerization and modulators of intracellular microfilaments.

### BRIEF DESCRIPTION OF THE DRAWINGS

For a more complete understanding of the features and advantages of the present invention, reference is now made to the detailed description of the invention along with the accompanying figures and in which:

FIG. 1A is an image of a gel illustrating the proteolytic cleavage of fibrocystin;

FIG. 1B is an image of a gel illustration mIMCD3/FC cells or mIMCD3/EGFP cells;

FIG. 1C is an image of an immunoblot analysis of nuclear extracts of mIMCD-3 cells (100 μg/lane);

FIGS. 2A, 2B and 2C are images of immuno-stained or autofluorescent cells;

FIGS. 3A, 3B and 3C are images of the identification of the nuclear localization signal (NLS) of fibrocystin;

FIGS. 4A, 4B 4C 4D, and 4E are images of intracellular Ca²⁺ release modulates fibrocystin cleavage;

FIGS. 5A, 5B, 5C and 5D are images that illustrate the activity of PKC modulates the cleavage of fibrocystin;

FIG. 6 illustrates the increased presence of cleavage products in the presence of the proteasome inhibitor MG-132;

FIG. 7 is an image of a gel that illustrates the cleavage of fibrocystin or stability of proteolytic fragments of fibrocystin can be modulated by affecting ATP-sensitive pathways and proteasome inhibitors and illustrates the increased presence of cleavage products in the presence of the proteasome inhibitor MG-132 in combination with ATP;

FIG. 8 is an image of a gel that illustrates the cleavage of fibrocystin can be altered with Calpain inhibitors;

FIG. 9 is an image of a gel illustrating the cleavage of fibrocystin or stability of proteolytic fragments of fibrocystin can be altered with the beta-secretase inhibitor Z-VLL-CHO;

FIG. 10 is a plot of the cleavage of fibrocystin or stability of proteolytic fragments of fibrocystin modulated by altering the degree of polymerization of microtubules, documented by usage of Taxol; and

FIG. 11 is a plot of the cleavage of fibrocystin or stability of proteolytic fragments of fibrocystin modulated by altering the degree of polarization of microtubules and microfilaments, documented by usage of Nocodazole, Latrunculin B, Cytochalasin D.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a method of reducing cyst formation in a patient with polycystic kidney disease by contacting the kidney of the patient with an effective amount of a fibrocystin cleavage inhibitor sufficient to reduce cyst formation. The fibrocystin cleavage inhibitor includes at least one of a proteasome inhibitor, a calpain inhibitor, and a β-secretase inhibitor and reduces the degradation of the fibrocystin cleavage products.

In some instances the fibrocystin cleavage inhibitor is a proteasome inhibitor selected from the group consisting of: MG-132 (Carbobenzoxy-L-leucyl-L-leucyl-L-leucinal), MG-115 (Carbobenzoxy-L-leucyl-L-leucyl-L-norvalinal), PSI (Carbobenzoxy-L-isoleucyl-γ-t-butyl-L-glutamyl-L-alanyl-L-leucinal), Lactacystin (Synthetic: N-Acetyl-L-Cysteine, S-[2R,3S,4R]-3-Hydroxy-2-[(1S)-1-Hydroxy-2-Methylpropyl-4-Methyl-5-Oxo-2-Pyrrolidinecarbonyl]), PS-519 (clasto-Lactacystin β-Lactone), α-Methylomuralide (α-Methyl clasto-Lactacystin β-Lactone), MG-101 (Ac-Leu-Leu-Nle-CHO), MG-262 (Z-Leu-Leu-Leu-B(OH)₂), PS-341 (Velcade; bortezomib), Epoxomicin ((2R)-2-[Acetyl-(N-Methyl-L-Isoleucyl)-L-Isoleucyl-L-Threonyl-L-Leucyl]-2-Methyloxirane), a calpain inhibitor selected from the group consisting of ritonavir, saquinavir, indinavir, nelfinavir, amprenavir, a β-secretase inhibitor is selected from Z-Val-Leu-Leu-CHO, and 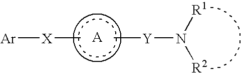
wherein Ar is an aromatic group; X is a divalent group selected from —O—, —S—, —CO—, —SO—, —SO₂—, —NR—, —CONR—, —SO₂NR—, and —COO— (wherein R1 is hydrogen, etc.), a divalent C1-6 aliphatic hydrocarbon group which may contain one or two of these divalent groups, or a bond; Y is a divalent group selected from —O—, —S—, —CO—, —SO—, —SO₂—, —NR—, —CONR—, —SO₂NR—, and —COO—, or a divalent C1-6 aliphatic hydrocarbon group which may contain one or two of these divalent groups; R and R2 are hydrogen, a hydrocarbon group, etc., respectively; and A is a ring which may be further substituted, or a salt thereof. The fibrocystin cleavage inhibitor may be a calpain inhibitor selected from Ac-Leu-Leu-Nle-H and Ac-Leu-Leu-Met-H. In addition, the calpain inhibitor selected may be selected from Boc-Phg-Asp-fmk, Boc-(2-F-Phg)-Asp-fmk, Boc-(F3-Val)-Asp-fmk, Boc-(3-F-Val)-Asp-fmk, Ac-Phg-Asp-fmk, Ac-(2-F-Phg)-Asp-fmk, Ac-(F3-Val)-Asp-fmk, Ac-(3-F-Val)-Asp-fmk, Z-Phg-Asp-fmk, Z-(2-F-Phg)-Asp-fmk, Z-(F3-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F3-Ala)-Asp-fmk, Z-(3-F-3-Me-Ala)-Asp-fmk, Z-(3-Cl-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF3-Ala)-Asp-fmk, Z-(4-CF3-Phg)-Asp-fmk, Z-(3-Me2 N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, or Z-(2-Thg)-Asp-fmk, wherein Boc is tert-butylcarbonyl, Phg is phenylglycine, fmk is fluoromethylketone, Z is benzyloxycarbonyl, Chg is cyclohexlglycine, Fug is furylglycine, Thg is thienylglycine, Fua is furylalanine, Tha is thienylalanine, Nal is naphthylalanine, Cha is cyclohexlalanine, Abu is aminobutyric acid, Tle is tert-leucine, Cpg is cyclopentylglycine, Cbg is cyclobutylglycine and Thz is thioproline.

In addition, the method may also include the step of contacting the kidney with one or more agents increase fibrocystin cleavage selected from modulators of microtubule polarization, modulators of Actin polymerization, modulators of contractile intracellular microfilaments and modulators of PKC activity. The agents are selected from Nocodazole, Benzolactam, Latrunculin, Cytochalasin B, Taxol and Bryostatin.

The present invention provides treatment of patients afflicted with polycystic kidney disease by regulation or stabilization of the proteolytic fragmentation of fibrocystin. The invention describes the alteration of proteolytic fibrocystin fragmentation by affecting intracellular Ca²⁺ concentration, protein kinase C (PKC) activation, cellular microfilaments organization, as well as application of proteinase inhibitors.

The autosomal dominant form of polycystic kidney disease is one of the most common hereditary diseases in man and is a frequent cause of end-stage renal disease. More than 1 in 1000 individuals carry mutations in either PKD1 or PKD2 and will develop renal cysts. Besides renal malformations, hepatic cysts arising from the biliary epithelia and cardiovascular manifestations are also commonly observed in autosomal recessive polycystic kidney disease. The gene products of PKD1 and PKD2, polycystin-1 and polycystin-2, are integral transmembrane proteins. The large N-terminal ectodomain of polycystin-1 contains an extensive array of protein domains implicated in protein-protein and protein-carbohydrate interaction followed by 11 membrane-spanning domains and a C-terminal cytoplasmic domain. The C-terminal tail of polycystin-1 interacts with the intracellular C-terminus of polycystin-2 via a coiled-coil domain and might form a functional complex (Qian, 1997; Tsiokas, 1997). Polycystin-2 has 6 transmembrane domains with intracellular C- and N-terminus. It functions as a non-selective Ca²⁺ permeant cation channel that can be activated by low concentrations of Ca²⁺.

The molecular pathogenesis of PKD may involve primary cilia and associated Ca²⁺ dependent signaling (Nauli, 2003). Primary cilia are present on the apical membrane of renal tubular epithelial cells, and bending of the cilia in response to fluid flow shear stress elicits an increase in cytosolic Ca²⁺ concentration (i.e., [Ca²⁺]). Polycystin-1 and polycystin-2, which are affected in the autosomal dominant form of PKD, are located in primary cilia and required for an initial Ca²⁺ influx that is triggered by fluid flow shear stress (Nauli, 2003).

Autosomal recessive polycystic kidney disease (ARPKD) is a hereditary cause of kidney failure in infants and children. Autosomal recessive polycystic kidney disease affects 1 in 20,000 individuals and is characterized by aberrant epithelial cell proliferation, which causes cystic dilation of the renal collecting ducts, and abnormal development of intrahepatic bile ducts (Lieberman, 1971). Affected individuals present with bilateral kidney enlargement, intrauterine kidney failure, and oligohydraminos; the latter causes pulmonary hypoplasia and limb and facial abnormalities. Children who survive the perinatal period or develop autosomal recessive polycystic kidney disease later in life develop chronic kidney disease and portal hypertension due to congenital hepatic fibrosis.

Autosomal recessive polycystic kidney disease is caused by mutations of the polycystic kidney and hepatic disease gene 1 (PKHD1) located on Chromosome 6 (Zerres, 1994). The protein encoded by PKHD1 is termed fibrocystin (also called polyductin, or tigmin (Xiong, 2002; Onuchic, 2002; Ward, 2002)) (Ward, 2002; Onuchic, 2002; Xiong, 2002). Fibrocystin is an approximately 500,000 Dalton type I membrane protein comprised of a large N-terminal ectodomain, a single transmembrane segment, and a short C-terminal cytoplasmic domain. The ectodomain contains arrays of IPT (Ig-like, plexins, transcription factors) domains and PbH1 (parallel beta-helix repeats) domains.

Fibrocystin is located in the primary cilium, as well as the basal body, which anchors the primary cilium in the cell body (Masyuk, 2003; Menezes, 2004; Wang, 2004; Ward, 2003). Together with observed similarities in disease manifestations, the overlapping subcellular localization of fibrocystin and polycystins, indicates a possible involvement in a common pathway.

The invention is based on the finding that fibrocystin is subjected to site-specific proteolytic cleavage. At least six different fragments that contain the C-terminus of fibrocystin are generated. Although the abundance of the larger proteolytic fragments varied under different conditions, a 21-kDa fragment (FCA) was consistently observed. The 21-kDa fragment contains most of the cytoplasmic domain of fibrocystin and was primarily observed in the nucleus. In contrast, full-length fibrocystin has been localized primarily in the primary cilium, basal body region, and apical plasma membrane and to a lesser degree in the cytoplasm. These findings suggest that fibrocystin undergoes regulated proteolysis releasing a cytoplasmic C-terminal fragment that translocates from the cilium, apical membrane, or cytoplasm to the nucleus.

Constitutive cleavage of fibrocystin was observed in culture systems in which primary cilia are formed. In contrast, no cleavage of fibrocystin was observed in cell culture systems that lack functional primary cilia. These findings suggest that fibrocystin proteolysis is functioning downstream of the ciliary signaling cascade; therefore, pharmacologic regulation of fibrocystin proteolysis can circumvent dysfunction of primary cilia and treat patients afflicted with polycystic kidney disease.

Reagents and Antibodies-Dantrolene, caffeine, ruthenium red, calphostin, thapsigargin, carbachol and PMA (phorbol 12-myristate 13-acetate) were from Sigma (St. Louis, Mo.). Mifepristone and anti-V5 antibody were from Invitrogen (Carlsbad, Calif.). Monoclonal mouse anti-fibrocystin antibody was described previously (Ward, 2003). To generate rabbit anti-fibrocystin serum (IgG8739), rabbits were immunized with a polyacrylamide gel-purified GST-fusion protein containing the cytoplasmic domain of mouse fibrocystin (amino acids 3869-4060).

Plasmids—cDNA encoding full-length (12,225 bp) human fibrocystin was assembled from 12 RT-PCR fragments that were synthesized using human kidney cDNA as template and verified by sequencing. A V5 epitope tag was attached in-frame to the C-terminus by removing the stop codon and inserting the DNA fragment into pcDNA3.1-V5/His (Invitrogen). The resultant plasmid was termed pFC-V5. The plasmid pGeneFC was created by inserting the fibrocystin coding region into pGene/V5 (Invitrogen). Inserting a cDNA encoding EGFP into pGene generated the plasmid pGeneEGFP. The plasmid pSwitch was from Invitrogen. To construct the plasmid pFCA-EGFP, a PCR fragment encoding the C-terminus of mouse fibrocystin (amino acids 3876-4059) was inserted 5′ to the EGFP coding region of pEGFP. Subsequently, the DNA fragment encoding FCA-EGFP was excised and inserted into pcDNA3.1. To construct the plasmid pFCA-DsRed, the EGFP coding region in pFCA-EGFP was replaced with a DNA fragment encoding DsRed2 (Clontech, Palo Alto, Calif.). The plasmids pEGFP-FC(3876-4059), pEGFP-FC(3876-3970), pEGFP-FC(3973-4031), pEGFP-FC(4043-4059), pEGFP-FC(3876-3940) pEGFP-FC3941-3970), pEGFP-FC(3946-3951), pEGFP-FC(3946-3954), pEGFP-FC(3941-3951), pEGFP-FC(3952-3970), pEGFP-FC(3949-3970), pEGFP-FC(3946-3970) and pEGFP-FC(3941-3963) were generated by inserting DNA encoding the corresponding regions of mouse fibrocystin into pEGFP-C3. To construct the plasmids pFC(3973-4031)-V5 and pFC(3875-3970)-V5, PCR fragments encoding the corresponding regions of mouse fibrocystin were cloned into pcDNA3.1-V5/His. The plasmid pLDLR-GV encoding the human low-density lipoprotein receptor (LDLR) fused at its C-terminus to Gal4-VP16 was a generous gift from Dr. Thomas Südhof (UT Southwestern, Dallas, Tex.). The plasmid pFC-GV was generated by inserting the Gal4-VP16 coding sequence into the ApaI site of human fibrocystin (at codon 3918). The pG5-luc reporter plasmid was a generous gift from Richard Baer (Columbia University, New York, N.Y.) (Yu, 1998). The plasmids pEF-neo and pEF-PKCαA/E were generous gifts from Gottfried Baier (Medical University of Innsbruck, Innsbruck, Austria).

Cell Culture, Transfection and Generation of Stable Cell Lines—mIMCD-3 cells were plated at a density of 500,000 cells/100 mm dish, and 24 hours and 48 hours later the cells were transfected with 2 μg of pFC-V5 using Effectene (Qiagen, Valencia, Calif.). Cells were incubated for an additional 72 hours and lysed in 100 μl buffer containing Tris-buffered saline, 0.5% Triton X-100, protease inhibitor cocktail (Hoffmann-La Roche Inc., Indianapolis, Ind.). Twenty μl of the lysates were analyzed by SDS-PAGE, and immunoblot analysis was performed using HRP-conjugated anti-V5 as described previously (Hiesberger, 2005). mIMCD3/FC and mIMCD3/EGFP cell lines with inducible expression of fibrocystin and EGFP, respectively, were produced by transfecting mIMCD-3 cells with 1.5 μg pSwitch and either 1.5 μg pGeneFC or 1.5 μg pGeneEGFP. Stable transfectants were isolated after 14 days of growth in media containing hygromycin (350 μg/ml) and zeocin (300 μg/ml). To test for inducible expression, cells derived from individual clones were treated with mifepristone (10 nM) for 48 h or left untreated, and proteins were analyzed by immunoblotting using HRP-conjugated anti-FLAG. HEK-293 cells stably transfected with the muscarinic acetylcholine receptor M3 were a generous gift from Trevor Shuttleworth (University of Rochester Medical center) (Yang, 1995).

Reporter Gene Assays—mIMCD-3 cells, HEK-293 cells, or HeLa cells were plated in 6-well dishes (3×10⁵ cells/well) and cotransfected with 0.05 μg phRL-TK(Int−) encoding Renilla luciferase (Promega, Madison, Wis.), 0.1 μg Photinus luciferase reporter plasmids, and 0.01-0.3 μg effector plasmids. Luciferase assays were performed as described previously (Hiesberger, 2004). Relative luciferase activity was calculated as the ratio of Photinus and Renilla luciferase.

Subcellular Fractionation—Fractions containing nuclear proteins, cell membrane proteins, or soluble proteins were generated essentially as described previously (DeBose-Boyd, 1999). Successful fractionation was confirmed by the distribution of the membrane protein polycystin-2 and the nuclear protein PCNA. To concentrate nuclear proteins, nuclear extract was precipitated with TCA.

Immunofluorescence and Microscopy-MDCK cells were grown in six well dishes and transfected with plasmids using Effectene. Twenty-four or 48 hours later, cells were fixed with acetone/methanol (1:1) and subjected to fluorescence microscopy. Paraffin embedded kidney sections were deparaffinated and rehydrated. Immunofluorescence was performed using anti-fibrocystin (2B) and Cy3-conjugated anti-mouse IgG (Molecular Probes). Images were obtained by deconvolution microscopy (Zeiss Axioplane-2, Openlab).

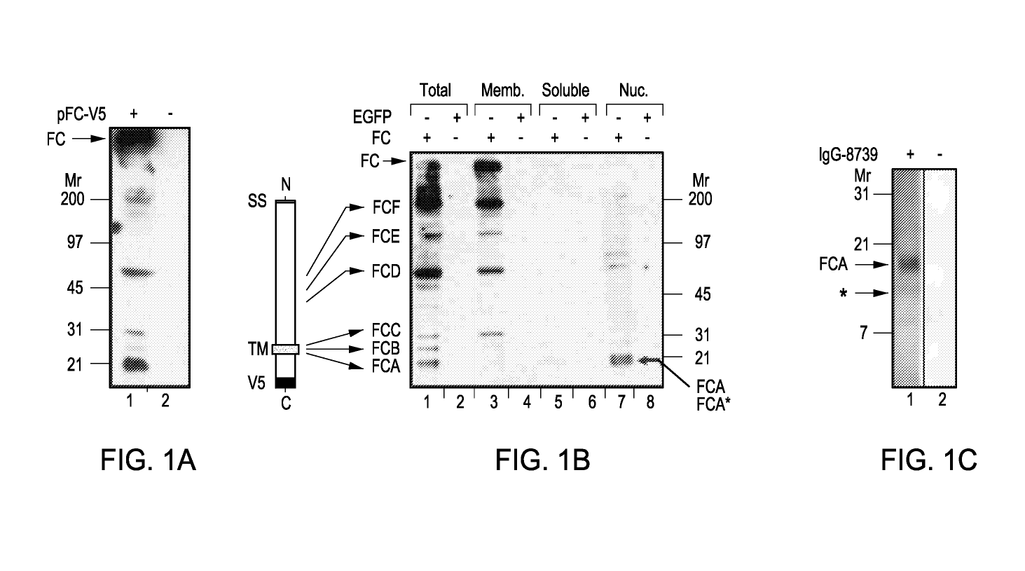

FIG. 1A is an image of a gel illustrating the proteolytic cleavage of fibrocystin generates a nuclear fragment mIMCD-3 cells were transfected with pFC-V5 (+) or empty pcDNA3.1 (−), and 72 h later whole cell lysates were analyzed by immunoblotting using anti-V5 antibody. Arrow indicates full-length fibrocystin (FC). FIG. 1B is an image of a gel illustrating mIMCD3/FC cells or mIMCD3/EGFP cells were grown for 4 days. Protein expression was induced with mifeprisone, and subcellular fractions were prepared 48 hours later. Ten percent of whole cell lysates (lanes 1 and 2), membrane fraction (lanes 3 and 4), cytosolic fraction (lanes 5 and 6), or 50% of nuclear fraction (lanes 7 and 8) were analyzed by immunoblotting using anti-V5 antibody. The structure of fibrocystin is shown schematically on the left, and arrows indicated estimated positions of cleavage sites. Where SS means signal sequence; TM means transmembrane segment; V5 means epitope tag; FCA-FCF means Fibrocystin fragment A-F. Asterisk (*) indicates that fragment FCA appeared as a doublet. FIG. 1C is an image of an immunoblot analysis of nuclear extracts of mIMCD-3 cells (100 μg/lane). Primary antibodies were rabbit polyclonal anti-fibrocystin IgG 8739 (lane 1) or control rabbit IgG (lane 2). Upper arrow indicates the FCA fragment. Asterisk (*) indicates a smaller fragment.

Proteolysis of fibrocystin produces a C-terminal nuclear fragment—A 12,225-bp DNA fragment encoding human fibrocystin was assembled from 12 RT-PCR fragments, cloned into the mammalian expression plasmid pcDNA3.1, and verified by sequencing. To facilitate detection of the recombinant protein, a C-terminal V5 epitope tag was added, and the resultant plasmid was termed pFC-V5. Mouse inner medullary collecting duct cells (mIMCD-3), which endogenously express native fibrocystin (Hiesberger, 2004), were transfected with pFC-V5 or empty pcDNA3.1, and cellular proteins were analyzed after 3 days. Immunoblotting utilizing an antibody directed against the C-terminal V5 epitope tag revealed a high molecular weight band corresponding to full-length fibrocystin as well as a series of proteolytic fragments as seen in FIG. 1A.

To further investigate the proteolytic cleavage of fibrocystin, mIMCD3/FC cells were generated, in which the expression of recombinant human fibrocystin can be induced by treatment with mifepristone. Immunoblot analysis of cells expressing recombinant fibrocystin revealed the presence of proteolytic fragments similar to those seen in transient transfection studies as seen in FIG. 1B.

The approximate sites of proteolytic cleavage of fibrocystin were estimated from the molecular weights of the proteolytic fragments. Since the cytoplasmic domain of human fibrocystin including the epitope tag has a calculated molecular weight of 25 kDa, the 21-kDa fragment (named FCA in FIG. 1B) is likely produced by cleavage in the cytoplasmic domain close to the transmembrane region. Proteolytic fragments of fibrocystin that retain the membrane-spanning segment as well as the C-terminal V5 epitope tag have a minimal molecular weight of 27.5 kDa, suggesting that fragments named FCC to FCF were generated by proteolytic cleavage in the ectodomain of fibrocystin. The site of cleavage producing the fragment named FCB may be within the transmembrane segment.

To determine the subcellular localization of the proteolytic fragments were prepared, e.g., membrane, cytosolic, and nuclear extracts and analyzed the proteins by immunoblotting. Fragments that were calculated to contain the transmembrane domain (FCC to FCF) were found in the membrane fraction, as expected (FIG. 1B). In contrast, the 21-kDa fragment containing the C-terminus of fibrocystin was detected in the nuclear fraction. This result suggests that the cytoplasmic domain of fibrocystin undergoes proteolytic cleavage releasing a 21-kDa fragment that translocates to the nucleus. In some studies, FCA appeared as well as doublet, suggesting that it may undergo additional cleavage or posttranslational modification (FCA and FCA* in FIG. 1B). The fragment FCB was found in the nuclear fraction and the membrane fraction but only after very long exposure of the fluorograms, suggesting that FCB is unstable or is lost during subcellular fractionation (data not shown).

To test whether endogenous fibrocystin undergoes proteolytic processing producing a nuclear fragment, nuclear extracts of mIMCD-3 cells were analyzed by immunoblotting. A polyclonal antibody raised against the C-terminal domain of fibrocystin recognized a protein corresponding in size to FCA (FIG. 1C). The 21-kDa fragment was detected using two different antibodies raised against the C-terminal domain of fibrocystin (not shown). Minor, smaller peptides were also detected, raising the possibility that endogenous FCA is subjected to further endoproteolytic processing (FIG. 1C, *).

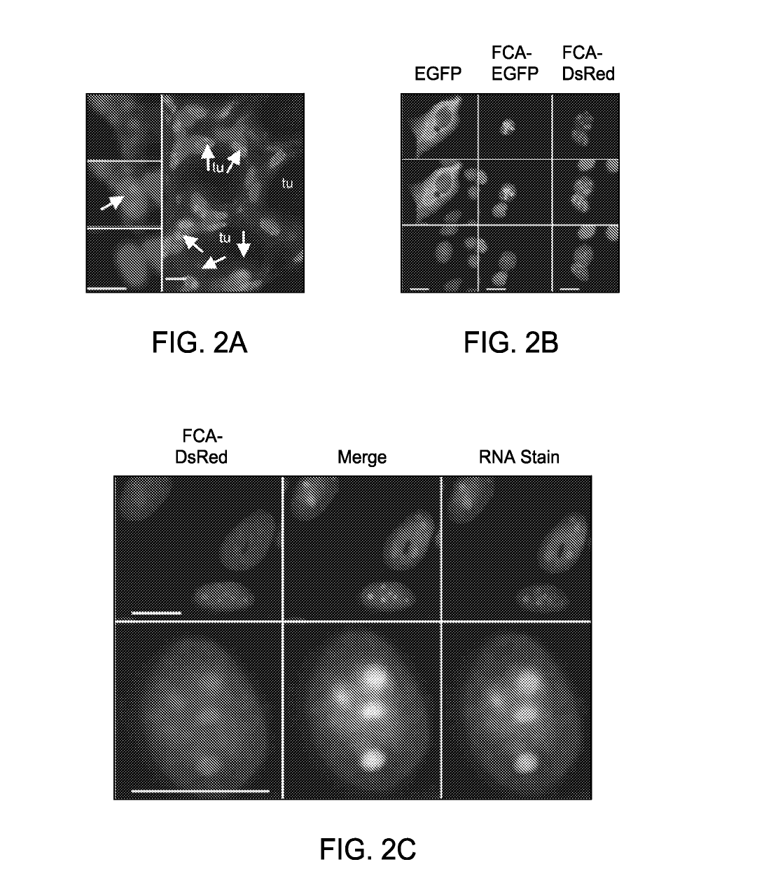

FIG. 2 is an image of the fibrocystin cytoplasmic domain is a nuclear protein. FIG. 2A is an image of section of P21 mouse kidney stained with anti-fibrocystin antibody (red). Nuclei were counterstained with DAPI (blue). Arrows indicate nuclear staining; tu, tubules; and Bars, 10 μm. FIG. 2B is an image that illustrates the MDCK cells transfected with plasmids encoding EGFP (green), EGFP fused to the cytoplasmic domain of fibrocystin (FCA-EGFP, green), or DsRed fused to the cytoplasmic domain of fibrocystin (FCA-DsRed, red). Nuclei were counterstained with DAPI (blue). FIG. 2C is an image of MDCK cells transfected with pFCA-DsRed (red), and nucleolar RNA was stained with RNASelect (green). Nuclei were counterstained with DAPI (blue).

Cytoplasmic domain of fibrocystin contains a nuclear localization signal—To determine the localization of the cytoplasmic domain of fibrocystin in vivo, kidney sections of 21-day old mice were stained with an antibody specific for the cytoplasmic region of the protein. Antibody staining confirmed that the cytoplasmic domain of fibrocystin was located in the nuclei of renal tubular epithelial cells (FIG. 2A). The cytoplasmic domain was also found in the cytosol as well as the primary cilia when the cells were observed in a different focal plane (data not shown). Interestingly, the fibrocystin staining in the nucleus exhibited a speckled pattern.

To define the mechanism of nuclear localization, the cytoplasmic domain of fibrocystin was linked to EGFP or DsRED and expressed in MDCK cells. The subcellular localization of the fusion proteins was determined by fluorescence microscopy. A recombinant protein containing the cytoplasmic domain of fibrocystin (amino acids 3876-4059) and EGFP was located exclusively in the nucleus, whereas EGFP by itself was predominantly in the cytosol (see FIG. 2B). Similarly, a fusion protein containing the cytoplasmic domain of fibrocystin and DsRED was located in the nucleus. Like endogenous fibrocystin, the fusion proteins were not diffusely distributed in the nucleus, but were concentrated in structures that had a speckled subnuclear distribution. To identity the nuclear structures, cells were transfected with plasmids encoding the cytoplasmic domain fused to DsRed, and the nucleoli were counterstained with an RNA-specific stain. Co-staining demonstrated that the fusion proteins containing the cytoplasmic domain were located in nucleoli (see FIG. 2C).

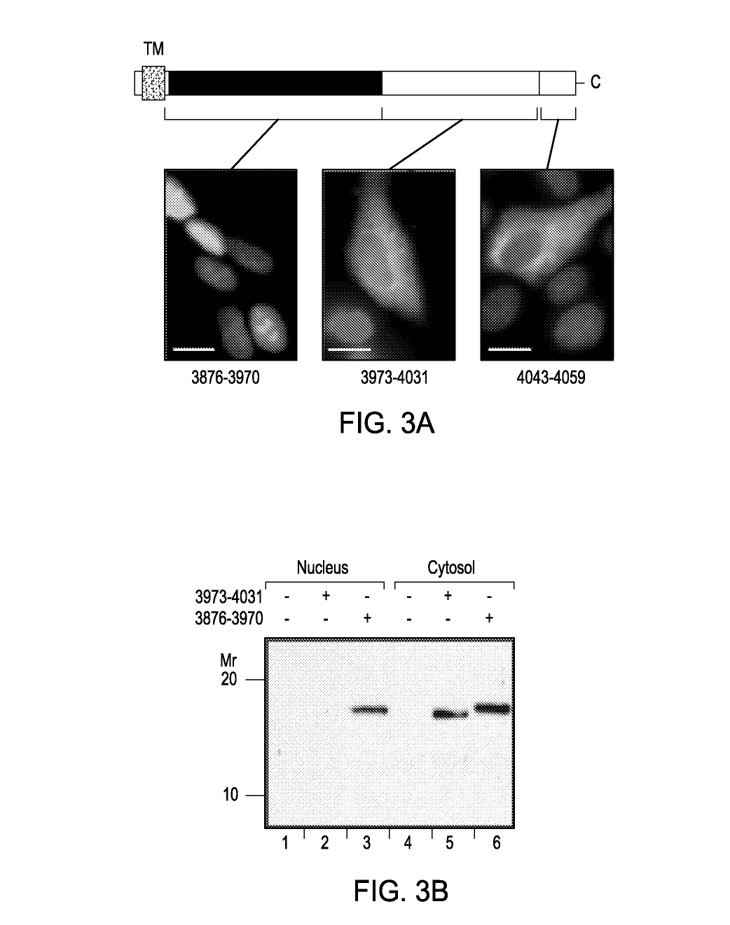

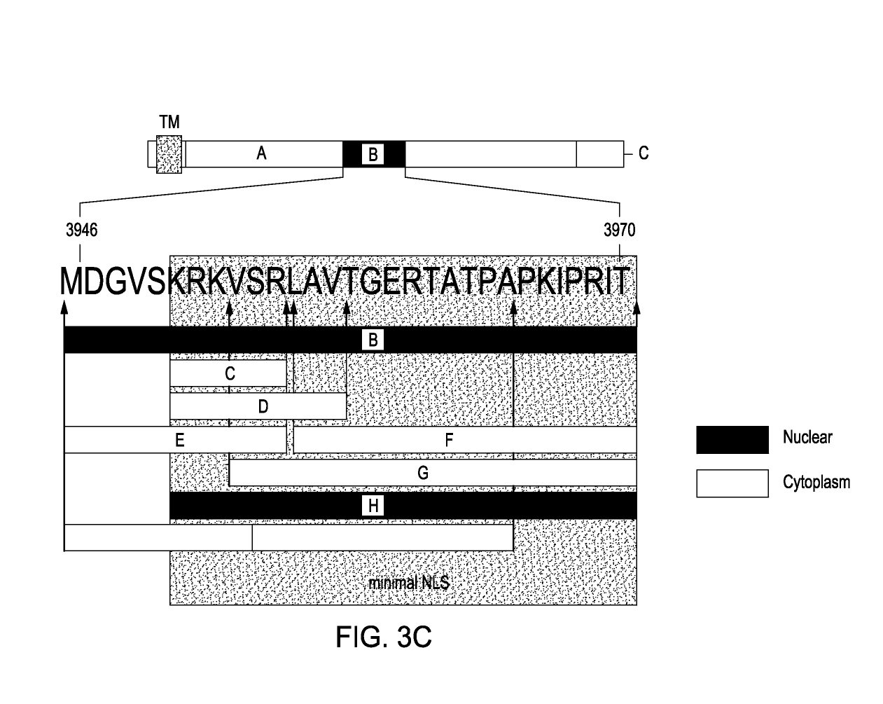

FIGS. 3A, 3B and 3C are images of the identification of the nuclear localization signal (NLS) of fibrocystin. FIG. 3A is an image of MCDK cells transfected with pEGFP-FC(3876-3970), pEGFP-FC(3973-4031), and pEGFP-FC(4043-4059) (green). Only EGFP-FC(3876-3970) is targeted to the nucleus (blue). FIG. 3B is an image of a gel illustrating HEK-293 cells transfected with pcDNA3.1, pFC(3973-4031)-V5, or pFC(3876-3970)-V5, and nuclear and cytosolic fractions were immunoblotted with anti-V5 antibody. FIG. 3C is a schematic illustrating MDCK cells transfected with plasmids encoding fusion proteins of EGFP and the indicated regions of fibrocystin. Subcellular localization of the fusion proteins was determined by fluorescence microscopy. Open bars indicate peptide sequences mediating cytoplasmic localization; closed bars indicate peptides that led to nuclear localization. Shaded box indicates the minimal nuclear localization signal (NLS).

To identify the region within the cytoplasmic domain of fibrocystin that is required for nuclear localization, plasmids encoding EGFP fused to the N-terminal portion (amino acids 3876-3970), the central portion (amino acids 3973-4031), and the C-terminal portion (amino acids 4043-4059) of the cytoplasmic domain were transfected into MDCK cells. Only an EGFP fusion protein containing the N-terminal portion was located in the nucleus, whereas fusion proteins containing the central or C-terminal portions remained in the cytosol (see FIG. 3A). To verify these findings using a different cell type and method, plasmids encoding V5 epitope-tagged N-terminal portion and central portion of the cytoplasmic domain were transfected into HEK-293 cells. Immunoblot analysis of nuclear and cytosolic fractions revealed that the N-terminal portion but not the central portion was present in the nuclear fraction (see FIG. 3B). To further define the sequence mediating nuclear localization, plasmids encoding various regions of the cytoplasmic domain of fibrocystin fused to EGFP were generated, and the subcellular localizations of the fusion proteins were evaluated. These studies defined the minimal nuclear localization signal (NLS) to be KRKVSRLAVTGERTATPAPKIPRIT (see FIG. 3C). Further deletions within this sequence abolished nuclear localization.

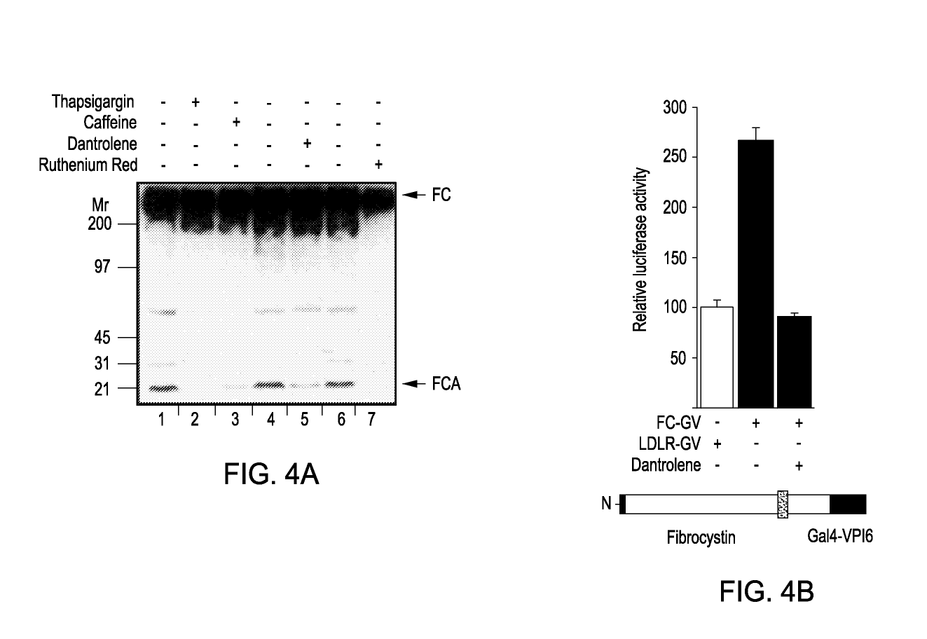

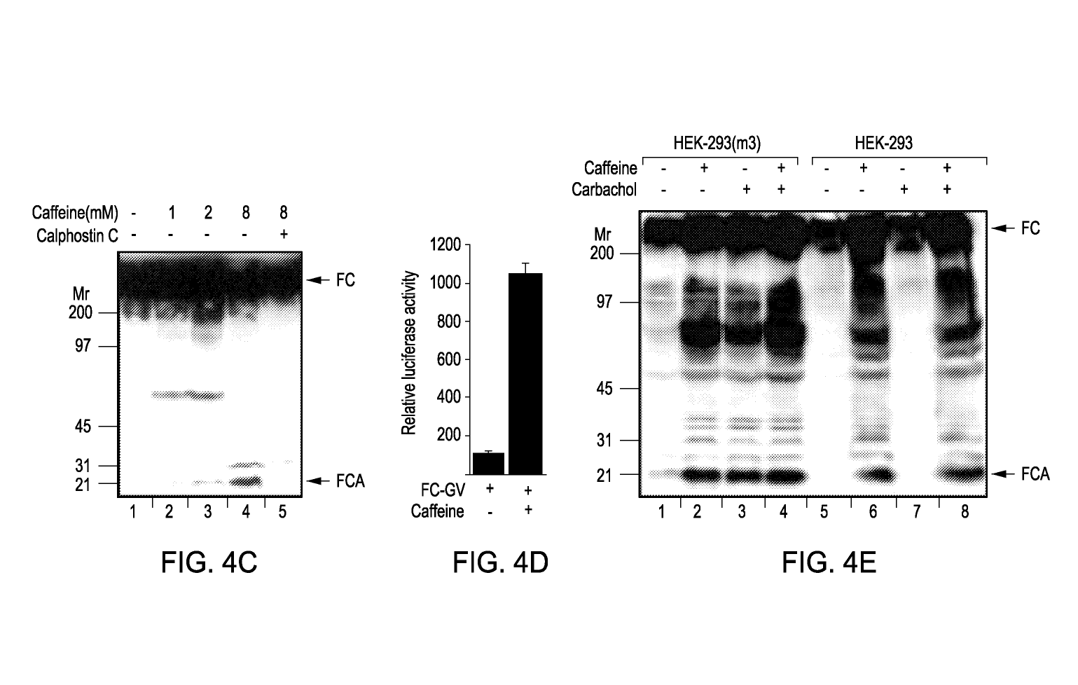

FIGS. 4A, 4B 4C 4D, and 4E are images of intracellular Ca²⁺ release modulates fibrocystin cleavage. FIG. 4 A is an image of a gel illustrating mIMCD3/FC cells were grown for 4 days and incubated with 0.1 μM thapsigargin (lane 2), 5 mM caffeine (lane 3), 75 μM dantrolene (lane 5), 200 μM ruthenium red (lane 7), or no additions (lanes 1, 4 and 6). After 2 hours, media were replaced with media lacking serum but containing the same supplements, and, in addition, 10 nM mifeprisone. Cell lysates were prepared after 24 hours and analyzed by immunoblotting using anti-V5 antibody. FIG. 4B is a graph of mIMCD-3 cells were transfected with 0.2 μg pG5-luc and either 0.2 μg pFC-GV or 0.2 μg pLDLR-GV. After 24 hours, cells were incubated in serum-free media in the presence or absence of 75 μM dantrolene. Relative luciferase activity was determined after 48 h. Data presented are mean ±SE of three separate transfections. The fibrocystin/Gal4-VP16 fusion protein is shown schematically below. Shaded box indicates the transmembrane segment; filled boxes indicate the N-terminal signal sequence and the C-terminal Gal4-VP16 fusion protein. FIG. 4C is an image of a gel illustrating HEK-293 cells transfected with 6 μg pFC-V5, and 8 hours later incubated in serum-free media containing 1-8 mM caffeine, 0.5 μM calphostin, or no additions. Cells were lysed after 16 h, and extracts were analyzed by immunoblotting using anti-V5 antibody. FIG. 4D is a graph of HEK-293 cells transfected with 0.2 μg pFC-GV and 0.2 μg pG5-luc. After 24 hours, cells were incubated in serum-free media containing 5 mM caffeine or no additions. Luciferase activity was measured after 12 hours. Data presented are mean ±SE of three separate transfections. FIG. 4E is an image of a gel illustrating HEK-293 cells stably expressing the m3 muscarinic receptor (lanes 1-4) and untransfected HEK-293 cells (lanes 5-8) were transfected with 6 μg pFC-V5. Eight h later the cells were incubated in serum-free media containing 5 mM caffeine (lanes 2, 4, 6 and 8), 100 μM carbachol (lanes 3, 4, 7 and 8), or no additions. Cells were lysed after 16 hours, and extracts were analyzed by immunoblotting using anti-V5 antibody.

Intracellular Ca²⁺ release is necessary and sufficient to trigger fibrocystin proteolysis—To determine whether the proteolytic cleavage of fibrocystin is affected by changes in intracellular Ca²⁺ concentration and Ca²⁺-induced Ca²⁺ release, mIMCD3/FC cells were pretreated with thapsigargin prior to induction of fibrocystin expression. Pretreatment with 0.1 μM thapsigargin to deplete intracellular Ca²⁺ stores (Young, 2004) abolished the generation of the FCA fragment (see FIG. 4A). Similarly, depletion of ryanodine-sensitive Ca²⁺ stores by pretreatment with 5 mM caffeine also prevented the generation of FCA (see FIG. 4A). Pretreatment with 200 μM ruthenium red, a nonspecific Ca²⁺ channel inhibitor, or treatment with 75 μM dantrolene, which interferes with ryanodine receptor (RyR)-mediated intracellular Ca²⁺ release, abolished the formation of FCA (FIG. 4A).

To quantitatively measure fibrocystin cleavage, a luciferase assay was developed that was similar to the ones employed to quantify the proteolytic cleavage of amyloid precursor protein (APP) and low density lipoprotein receptor related protein-1 (LRP1) (Cao, 2001; May, 2003). A plasmid (FC-GV) encoding the synthetic transcription factor Gal4-VP16 fused to the C-terminus of fibrocystin was generated. Proteolytic cleavage of the fibrocystin fusion protein releases Gal4-VP16, which translocates to the nucleus and activates a Gal4-responsive luciferase reporter gene (pG5-luc). Therefore, luciferase expression correlates with fibrocystin cleavage. Transfection of mIMCD-3 cells with a plasmid encoding FC-GV stimulated luciferase activity 2.6-fold compared with cells expressing LDLR-GV (low-density lipoprotein receptor fused to Gal4-VP16), which is not subjected to proteolysis. The increase in luciferase activity was abolished in the presence of dantrolene, verifying that RyR activity was required for fibrocystin cleavage (see FIG. 4B).

To test whether intracellular Ca²⁺ release is not only required but is also sufficient to induce fibrocystin proteolysis, a short-term cell culture system of human embryonic kidney cells (HEK-293) was used. These cells express RyR and exhibit a RyR-mediated increase of intracellular Ca²⁺ upon treatment with caffeine (Querfurth, 1998). Under basal conditions, no proteolytic fragments of transiently transfected fibrocystin were detected. However, addition of caffeine induced dose-dependent proteolysis of fibrocystin (see FIG. 4C). Caffeine-induced fibrocystin proteolysis was prevented by treatment with the protein kinase C(PKC) inhibitor calphostin C. This latter result indicated a possible role of PKC in the Ca²⁺-induced cleavage of fibrocystin.

To quantify fibrocystin proteolysis in response to intracellular Ca²⁺ release, the luciferase reporter assay was employed. HEK-293 cells were cotransfected with the plasmids pFC-GV and pG5-luc and incubated with either 5 mM caffeine or vehicle (as a control). Treatment with caffeine increased luciferase activity 10-fold confirming that pharmacological stimulation of intracellular Ca²⁺ release was sufficient to induce fibrocystin cleavage (see FIG. 4D).

Next, whether fibrocystin cleavage could be induced by activation of a cell surface receptor was tested to stimulates intracellular Ca²⁺ release. These studies utilized HEK-293 cells stably expressing the muscarinic acetylcholine receptor m3 [HEK-293(m3)], which respond to treatment with the agonist carbachol with activation of PLC and IP3-mediated Ca²⁺ release (Schmidt, 1994). Exposure to 5 mM caffeine triggered fibrocystin proteolysis in both HEK-293 cells and HEK-293(m3) cells, whereas treatment with 100 μM carbachol induced fibrocystin cleavage only in HEK-293(m3) cells (see FIG. 4E). Taken together, these results demonstrate that stimulation of intracellular Ca²⁺ release is sufficient to trigger fibrocystin cleavage.

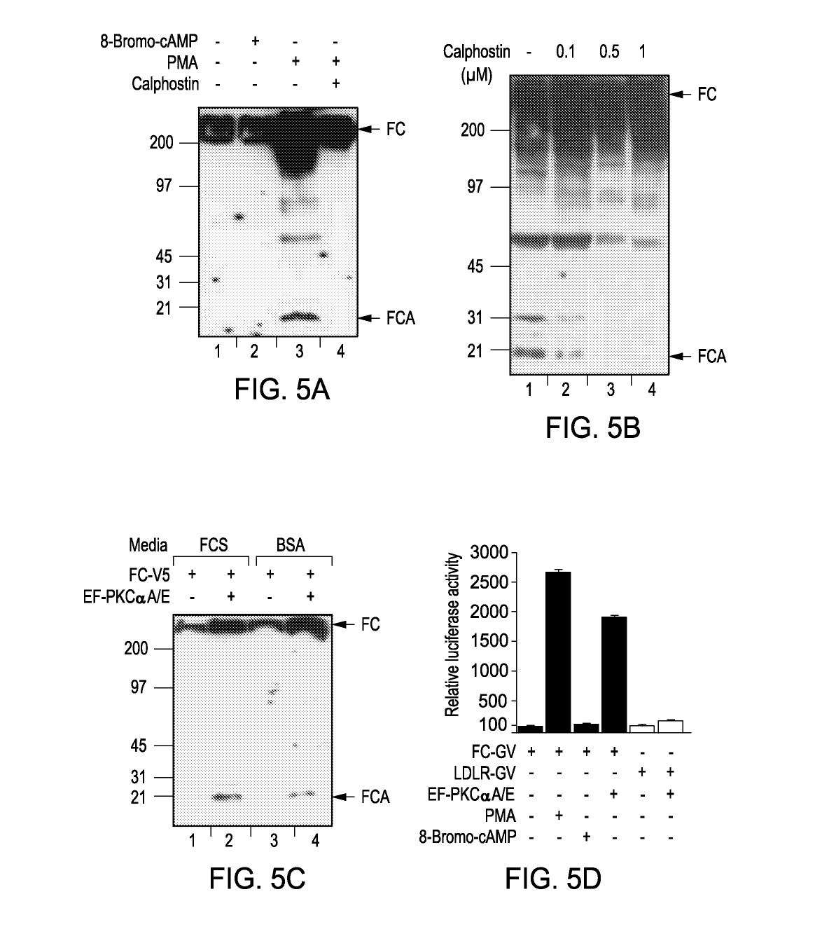

FIGS. 5A, 5B, 5C and 5D are images that illustrate the activity of PKC modulates the cleavage of fibrocystin. FIG. 5A is an image of a gel that illustrates HEK-293 cells were transfected with 6 μg pFC-V5. After 8 hours, media were supplemented with 500 μM 8-Bromo-cAMP, 100 nM PMA, or 0.5 μM calphostin C. Fourteen hours later, cellular proteins were extracted and analyzed by immunoblotting using anti-V5 antibody. FIG. 5B is an image of a gel mIMCD3/FC cells were grown for 4 days, and expression of fibrocystin was induced by addition of 10 nM mifepristone. After 24 hours, media were supplemented with 0.1, 0.5, or 1 μM calphostin C. Thirty hours later, cells were harvested, and proteins were analyzed by immunoblotting using anti-V5 antibody. FIG. 5C is an image of a gel illustrating HEK-293 cells were transfected with 3 μg pFC-V5 and either 3 μg of the empty vector pEF-neo (lanes 1 and 3) or 3 μg pEF-PKCαA/E (lanes 2 and 4). After 12 hours media were changed to media containing 10% FCS (lanes 1 and 2) or 0.2% BSA (lanes 3 and 4). Forty-eight hours later, cells were harvested, and proteins were subjected to immunoblotting using anti-V5 antibody. FIG. 5D is a plot illustrating HEK-293 cells transfected with 0.2 μg pG5-luc and 0.2 μg pFC-GV, pLDLR-GV and/or pEF-PKCαA/E. After 16 hours, media were changed to serum-free media containing 100 nM PMA or 500 μM Br-cAMP. Twenty-four hours later, cells were harvested, and relative luciferase activities were determined. Data are the mean ±SE of three independent transfections.

Activation of PKC is necessary and sufficient for the generation of FCA—The inhibition of fibrocystin proteolysis in the presence of the PKC inhibitor calphostin C suggested an involvement of members of the group of conventional PKC (PKCα, PKCβ1, PKCβ2 or PKCγ). To test whether activation of PKC is sufficient to induce fibrocystin cleavage, HEK-293 cells were transfected with pFC-V5 and exposed to activators of protein kinases. Treatment with PMA, an activator of PKC, produced a marked increase of proteolytic cleavage, which was prevented by pretreatment with calphostin C (see FIG. 5A). In contrast, no cleavage was observed when cells were left untreated or treated with the protein kinase A (PKA) activator 8-Br-cAMP. However, besides inducing proteolysis, PMA also increased the total expression of fibrocystin, controlled by the CMV promoter, which made it difficult to unequivocally correlate PKC activation with fibrocystin cleavage.

To address this issue, two additional approaches were used: (i) replacement of the PKC-responsive CMV-promoter, and (ii) genetic activation of PKC. mIMCD3/FC cells, in which fibrocystin is expressed under the control of a meifepristone-responsive promoter, were treated with calphostin C 24 hours after induction, and cleavage was analyzed 30 hours later. Treatment with Calphostin C did not alter expression levels of fibrocystin but produced a dose-dependent inhibition of fibrocystin proteolysis (see FIG. 5B). Next, a constitutively-active pseudosubstrate mutant of PKCα (PKCαA/E) (Baier-Bitterlich, 1996) was used. Cotransfection of a plasmid encoding PKCαA/E resulted in fibrocystin proteolysis and generation of FCA, whereas cotransfection of empty expression plasmid did not affect processing (see FIG. 5C). The presence or absence of fetal calf serum (FCS) did not markedly affect cleavage.

To quantify the cleavage of fibrocystin stimulated by PKC activation, luciferase reporter assays were performed. HEK-293 cells were cotransfected with pFC-GV and pG5-luc and PKC activity was stimulated by treatment with PMA or expression of the PKCαA/E mutant. Fibrocystin proteolysis was evaluated by measuring luciferase activity. Treatment with PMA increased luciferase activity 27-fold, whereas the addition of 8-Br-cAMP did not significantly affect luciferase activity. Co-expression of PKCαA/E stimulated luciferase activity 19-fold but had no effect on cells expressing the negative control LDLR-GV (see FIG. 5D). Taken together, these results demonstrate that fibrocystin-specific proteolysis is PKC-dependent. In addition, the cleavage of fibrocystin or stability of proteolytic fragments of fibrocystin can be modulated using the PKC modulator Bryostatin and Benzolactam.

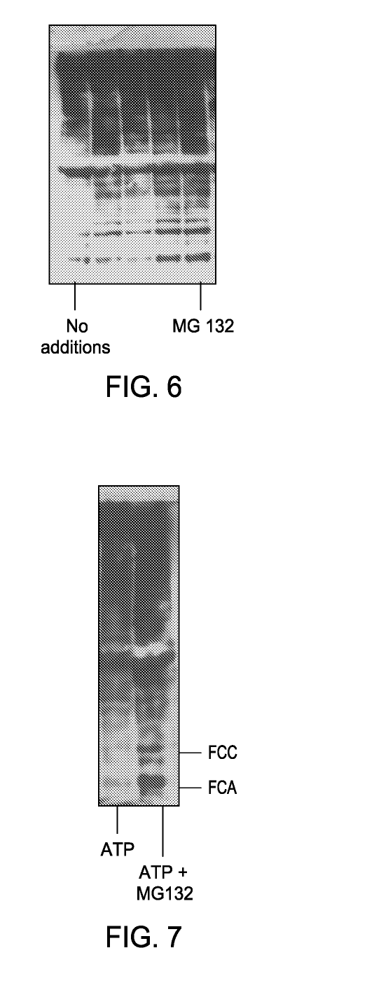

FIG. 6 is an image of a gel illustrating the cleavage of fibrocystin or stability of proteolytic fragments of fibrocystin can be modulated by inhibitors of the proteasome; MG-132. FIG. 6 illustrates the increased presence of cleavage products in the presence of the proteasome inhibitor MG-132. FIG. 6 illustrates that the cleavage of fibrocystin or stability of proteolytic fragments of fibrocystin can be modulated by inhibitors of the proteasome; MG-132. The figure shows an increase of fibrocystin cleavage products in the presence of the proteasome inhibitor MG-132. mIMCD3/FC cells were grown for 4 days, and expression of fibrocystin was induced by addition of 10 nM mifepristone. After 24 hours, media were supplemented with vehicle or 2 μM MG-132. Thirty hours later, cells were harvested, and proteins were analyzed by immunoblotting using anti-V5 antibody.

FIG. 7 is an image of a gel that illustrates the cleavage of fibrocystin or stability of proteolytic fragments of fibrocystin can be modulated by affecting ATP-sensitive pathways and proteasome inhibitors and illustrates the increased presence of cleavage products in the presence of the proteasome inhibitor MG-132 in combination with ATP. FIG. 7 illustrates that the cleavage of fibrocystin or stability of proteolytic fragments of fibrocystin can be modulated by affecting ATP-sensitive pathways and proteasome inhibitors. The figure shows the increased presence of cleavage products in the presence of the proteasome inhibitor MG-132 in combination with ATP. mIMCD3/FC cells were grown for 4 days, and expression of fibrocystin was induced by addition of 10 nM mifepristone. After 24 hours, media were supplemented with 20 μM ATP and either vehicle or 2 μM MG-132. Thirty hours later, cells were harvested, and proteins were analyzed by immunoblotting using anti-V5 antibody.

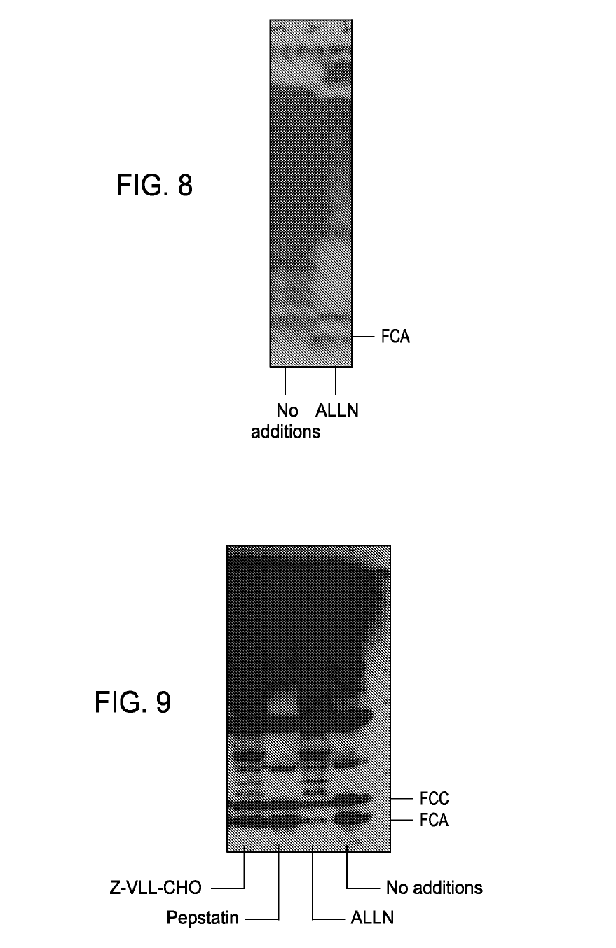

FIG. 8 is an image of a gel that illustrates the cleavage of fibrocystin or stability of proteolytic fragments of fibrocystin can be altered with Calpain inhibitors; ALLN. FIG. 8 illustrates that the cleavage of fibrocystin or stability of proteolytic fragments of fibrocystin can be altered with Calpain inhibitors; ALLN. The figure shows alteration of the abundance of fibrocystin proteolytic fragments by the presence of ALLN. HEK-293 cells were transfected with 6 μg pFC-V5. After 8 hours, media were supplemented with vehicle or 10 μM ALLN. Fourteen hours later, cellular proteins were extracted and analyzed by immunoblotting using anti-V5 antibody.

While FIG. 9 is an image of a gel illustrating the cleavage of fibrocystin or stability of proteolytic fragments of fibrocystin can be altered with the beta-secretase inhibitor Z-VLL-CHO. FIG. 9 illustrates that the cleavage of fibrocystin or stability of proteolytic fragments of fibrocystin can be altered with the beta-secretase inhibitor Z-VLL-CHO or ALLN. mIMCD3/FC cells were grown for 4 days, and expression of fibrocystin was induced by addition of 10 nM mifepristone. After 24 hours, media were supplemented with vehicle, 15 μM Z-VLL-CHO, pepstatin or 10 μM ALLN. Thirty hours later, cells were harvested, and proteins were analyzed by immunoblotting using anti-V5 antibody.

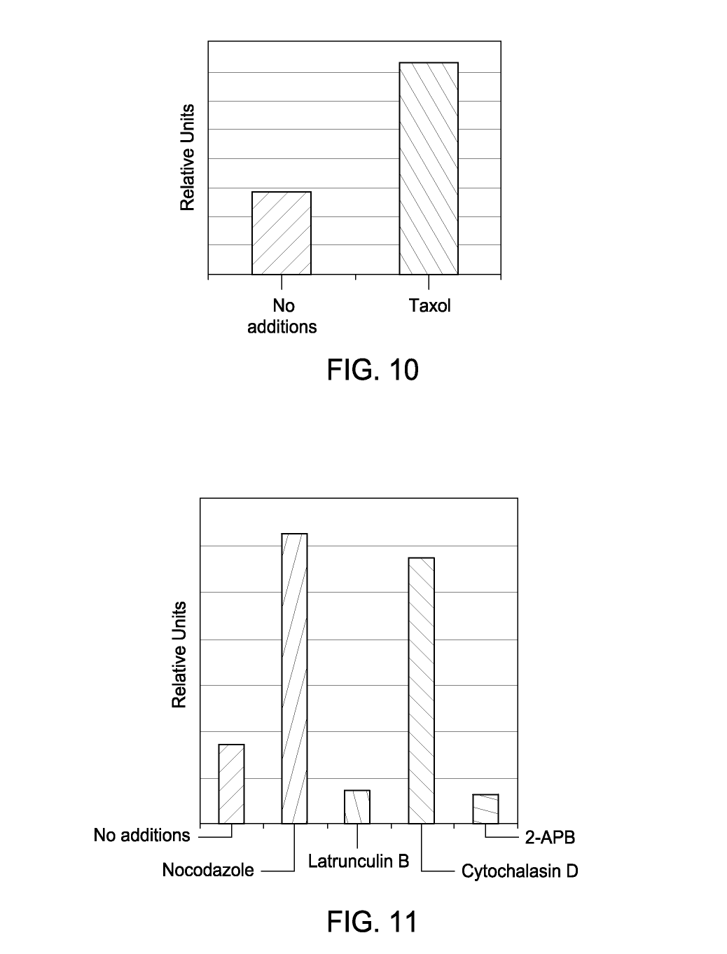

FIG. 10 is a plot of the cleavage of fibrocystin or stability of proteolytic fragments of fibrocystin can be modulated by altering the degree of polymerization of microtubules, documented by usage of Taxol. HEK-293 cells were transfected with 0.2 μg pFC-GV and 0.2 μg pG5-luc. After 24 hours, cells were incubated in serum-free media containing Taxol or in serum-free media without additions. Luciferase activity was measured after 12 hours.

FIG. 11 is a plot of the cleavage of fibrocystin or stability of proteolytic fragments of fibrocystin can be modulated by altering the degree of polarization of microtubules, documented by usage of Nocodazole; by altering the degree of Actin polymerization, documented by usage of Latrunculin B; by altering the formation of contractile intracellular microfilaments, documented by usage of Cytochalasin B; as well as by the IP3 channel inhibitor 2-APB. HEK-293 cells were transfected with 0.2 μg pFC-GV and 0.2 μg pG5-luc. After 24 hours, cells were incubated in serum-free media containing 10 μg/ml Nocodazole, 2 μg/ml Latrunculin, 10 μM Cytochalasin B, 50 μg/ml 2-APB, or in serum-free media without additions. Luciferase activity was measured after 12 hours.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Baier-Bitterlich, G., Uberall, F., Bauer, B., Fresser, F., Wachter, H., Grunicke, H., Utermann, G., Altman, A., and Baier, G. (1996). Protein kinase C-theta isoenzyme selective stimulation of the transcription factor complex AP-1 in T lymphocytes. Mol Cell Biol 16, 1842-1850.
Cai, Y., Maeda, Y., Cedzich, A., Torres, V. E., Wu, G., Hayashi, T., Mochizuki, T., Park, J. H., Witzgall, R., and Somlo, S. (1999). Identification and characterization of polycystin-2, the PKD2 gene product. J Biol Chem 274, 28557-28565.
Cao, X., and Sudhof, T. C. (2001). A transcriptionally [correction of transcriptively] active complex of APP with Fe65 and histone acetyltransferase Tip60. Science 293, 115-120.
Chauvet, V., Tian, X., Husson, H., Grimm, D. H., Wang, T., Hieseberger, T., Igarashi, P., Bennett, A. M., Ibraghimov-Beskrovnaya, O., Somlo, S., and Caplan, M. J. (2004). Mechanical stimuli induce cleavage and nuclear translocation of the polycystin-1 C terminus. J Clin Invest 114, 1433-1443.
DeBose-Boyd, R. A., Brown, M. S., Li, W. P., Nohturfft, A., Goldstein, J. L., and Espenshade, P. J. (1999). Transport-dependent proteolysis of SREBP: relocation of site-1 protease from Golgi to ER obviates the need for SREBP transport to Golgi. Cell 99, 703-712.
Eley, L., Yates, L. M., and Goodship, J. A. (2005). Cilia and disease. Curr Opin Genet Dev 15, 308-314.
Etcheberrigaray, R., Tan, M., Dewachter, I., Kuiperi, C., Van der Auwera, I., Wera, S., Qiao, L., Bank, B., Nelson, T. J., Kozikowski, A. P., et al. (2004). Therapeutic effects of PKC activators in Alzheimer's disease transgenic mice. Proc Natl Acad Sci USA 101, 11141-11146.
Fan, S., Hurd, T. W., Liu, C. J., Straight, S. W., Weimbs, T., Hurd, E. A., Domino, S. E., and Margolis, B. (2004). Polarity proteins control ciliogenesis via kinesin motor interactions. Curr Biol 14, 1451-1461.
Hiesberger, T., Bai, Y., Shao, X., McNally, B. T., Sinclair, A. M., Tian, X., Somlo, S., and Igarashi, P. (2004). Mutation of hepatocyte nuclear factor-1beta inhibits Pkhd1 gene expression and produces renal cysts in mice. J Clin Invest 113, 814-825.
Hiesberger, T., Shao, X., Gourley, E., Reimann, A., Pontoglio, M., and Igarashi, P. (2005). Role of the hepatocyte nuclear factor-beta (HNF-beta) C-terminal domain in Pkhd1 (ARPKD) gene transcription and renal cystogenesis. J Biol. Chem.
Igarashi, P., and Somlo, S. (2002). Genetics and pathogenesis of polycystic kidney disease. J Am Soc Nephrol 13, 2384-2398.
Jans, D. A., Xiao, C. Y., and Lam, M. H. (2000). Nuclear targeting signal recognition: a key control point in nuclear transport? Bioessays 22, 532-544.
Koulen, P., Cai, Y., Geng, L., Maeda, Y., Nishimura, S., Witzgall, R., Ehrlich, B. E., and Somlo, S. (2002). Polycystin-2 is an intracellular calcium release channel. Nat Cell Biol 4, 191-197.
Krause, T., Gerbershagen, M. U., Fiege, M., Weisshorn, R., and Wappler, F. (2004). Dantrolene—a review of its pharmacology, therapeutic use and new developments. Anaesthesia 59, 364-373.
Landman, N., and Kim, T. W. (2004). Got RIP? Presenilin-dependent intramembrane proteolysis in growth factor receptor signaling. Cytokine Growth Factor Rev 15, 337-351.
Leung, A. K., and Lamond, A. I. (2003). The dynamics of the nucleolus. Crit. Rev Eukaryot Gene Expr 13, 39-54.
Masyuk, T. V., Huang, B. Q., Ward, C. J., Masyuk, A. I., Yuan, D., Splinter, P. L., Punyashthiti, R., Ritman, E. L., Torres, V. E., Harris, P. C., and LaRusso, N. F. (2003). Defects in cholangiocyte fibrocystin expression and ciliary structure in the PCK rat. Gastroenterology 125, 1303-1310.
May, P., Bock, H. H., Nimpf, J., and Herz, J. (2003). Differential glycosylation regulates processing of lipoprotein receptors by gamma-secretase. J Biol Chem 278, 37386-37392.
Menezes, L. F., Cai, Y., Nagasawa, Y., Silva, A. M., Watkins, M. L., Da Silva, A. M., Somlo, S., Guay-Woodford, L. M., Germino, G. G., and Onuchic, L. F. (2004). Polyductin, the PKHD1 gene product, comprises isoforms expressed in plasma membrane, primary cilium, and cytoplasm. Kidney Int 66, 1345-1355.
Nauli, S. M., Alenghat, F. J., Luo, Y., Williams, E., Vassilev, P., Li, X., Elia, A. E., Lu, W., Brown, E. M., Quinn, S. J., et al. (2003). Polycystins 1 and 2 mediate mechanosensation in the primary cilium of kidney cells. Nat Genet 33, 129-137.
Onuchic, L. F., Furu, L., Nagasawa, Y., Hou, X., Eggermann, T., Ren, Z., Bergmann, C., Senderek, J., Esquivel, E., Zeltner, R., et al. (2002). PKHD1, the polycystic kidney and hepatic disease 1 gene, encodes a novel large protein containing multiple immunoglobulin-like plexin-transcription-factor domains and parallel beta-helix 1 repeats. Am J Hum Genet 70, 1305-1317.
Ozawa, T. (2001). Ryanodine-sensitive Ca²⁺ release mechanism in non-excitable cells (Review). Int J Mol Med 7, 21-25.
Qian, F., Germino, F. J., Cai, Y., Zhang, X., Somlo, S., and Germino, G. G. (1997). PKD 1 interacts with PKD2 through a probable coiled-coil domain. Nat Genet 16, 179-183.
Querfurth, H. W., Haughey, N. J., Greenway, S. C., Yacono, P. W., Golan, D. E., and Geiger, J. D. (1998). Expression of ryanodine receptors in human embryonic kidney (HEK293) cells. Biochem J 334 (Pt 1), 79-86.
Schmidt, M., Huwe, S. M., Fasselt, B., Homann, D., Rumenapp, U., Sandmann, J., and Jakobs, K. H. (1994). Mechanisms of phospholipase D stimulation by m3 muscarinic acetylcholine receptors. Evidence for involvement of tyrosine phosphorylation. Eur J Biochem 225, 667-675.
Tsiokas, L., Kim, E., Arnould, T., Sukhatme, V. P., and Walz, G. (1997). Homo- and heterodimeric interactions between the gene products of PKD1 and PKD2. Proc Natl Acad Sci USA 94, 6965-6970.
Wang, S., Luo, Y., Wilson, P. D., Witman, G. B., and Zhou, J. (2004). The autosomal recessive polycystic kidney disease protein is localized to primary cilia, with concentration in the basal body area. J Am Soc Nephrol 15, 592-602.
Ward, C. J., Hogan, M. C., Rossetti, S., Walker, D., Sneddon, T., Wang, X., Kubly, V., Cunningham, J. M., Bacallao, R., Ishibashi, M., et al. (2002). The gene mutated in autosomal recessive polycystic kidney disease encodes a large, receptor-like protein. Nat Genet 30, 259-269.
Ward, C. J., Yuan, D., Masyuk, T. V., Wang, X., Punyashthiti, R., Whelan, S., Bacallao, R., Torra, R., LaRusso, N. F., Torres, V. E., and Harris, P. C. (2003). Cellular and subcellular localization of the ARPKD protein; fibrocystin is expressed on primary cilia. Hum Mol Genet 12, 2703-2710.
Xiong, H., Chen, Y., Yi, Y., Tsuchiya, K., Moeckel, G., Cheung, J., Liang, D., Tham, K., Xu, X., Chen, X. Z., et al. (2002). A novel gene encoding a TIG multiple domain protein is a positional candidate for autosomal recessive polycystic kidney disease. Genomics 80, 96-104.
Yang, J., Williams, J. A., Yule, D. I., and Logsdon, C. D. (1995). Mutation of carboxyl-terminal threonine residues in human m3 muscarinic acetylcholine receptor modulates the extent of sequestration and desensitization. Mol Pharmacol 48, 477-485.
Young, H. S., and Stokes, D. L. (2004). The mechanics of calcium transport. J Membr Biol 198, 55-63.
Yu, X., Wu, L. C., Bowcock, A. M., Aronheim, A., and Baer, R. (1998). The C-terminal (BRCT) domains of BRCA1 interact in vivo with CtIP, a protein implicated in the CtBP pathway of transcriptional repression. J Biol Chem 273, 25388-25392.

What is claimed is:

1. A method of reducing cyst formation in a patient with polycystic kidney disease comprising the step of:
contacting the kidney of the patient with an effective amount of a fibrocystin cleavage inhibitor sufficient to reduce cyst formation, wherein the fibrocystin cleavage inhibitor comprises at least one of a proteasome inhibitor, a calpain inhibitor, and a β-secretase inhibitor and reduces the degradation of the fibrocystin cleavage products.

2. The method of claim 1, wherein the fibrocystin cleavage inhibitor is a proteasome inhibitor selected from the group consisting of: MG-132 (Carbobenzoxy-L-leucyl-L-leucyl-L-leucinal), MG-115 (Carbobenzoxy-L-leucyl-L-leucyl-L-norvalinal), PSI (Carbobenzoxy-L-isoleucyl-γ-t-butyl-L-glutamyl-L-alanyl-L-leucinal), Lactacystin (Synthetic: N-Acetyl-L-Cysteine, S-[2R,3 S,4R]-3-Hydroxy-2-[(1S)-1-Hydroxy-2-Methylpropyl-4-Methyl-5-Oxo-2-Pyrrolidinecarbonyl]), PS-519 (clasto-Lactacystin β-Lactone), α-Methylomuralide (α-Methyl clasto-Lactacystin β-Lactone), MG-101 (Ac-Leu-Leu-Nle-CHO), MG-262 (Z-Leu-Leu-Leu-B(OH)₂), PS-341 (Velcade; bortezomib), and Epoxomicin ((2R)-2-[Acetyl-(N-Methyl-L-Isoleucyl)-L-Isoleucyl-L-Threonyl-L-Leucyl]-2-Methyloxirane).

3. The method of claim 1, wherein fibrocystin cleavage inhibitor is MG-132, MG-115 or MG-101 and the inhibition of proteosome activity causes an increase of Fibrocystin A fragment.

4. The method of claim 1, wherein the fibrocystin cleavage inhibitor is a calpain inhibitor selected from the group consisting of ritonavir, saquinavir, indinavir, nelfinavir, amprenavir.

5. The method of claim 1, wherein the fibrocystin cleavage inhibitor is a β-secretase inhibitor is selected from Z-Val-Leu-Leu-CHO, and 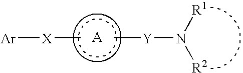
wherein Ar is an aromatic group; X is a divalent group selected from —O—, —S—, —CO—, —SO—, —SO2-, —NR—, —CONR—, —SO₂NR—, and —COO— (wherein R¹ is hydrogen, etc.), a divalent C1-6 aliphatic hydrocarbon group which may contain one or two of these divalent groups, or a bond; Y is a divalent group selected from —O—, —S—, —CO—, —SO—, —SO₂—, —NR—, —CONR—, —SO₂NR—, and —COO—, or a divalent C1-6 aliphatic hydrocarbon group which may contain one or two of these divalent groups; R and R² are hydrogen, a hydrocarbon group, etc., respectively; and A is a ring which may be further substituted, or a salt thereof.

6. The method of claim 1, wherein fibrocystin cleavage inhibitor is a calpain inhibitor selected from Ac-Leu-Leu-Nle-H and Ac-Leu-Leu-Met-H.

7. The method of claim 1, wherein fibrocystin cleavage inhibitor is a calpain inhibitor selected from Boc-Phg-Asp-fmk, Boc-(2-F-Phg)-Asp-fmk, Boc-(F3-Val)-Asp-fmk, Boc-(3-F-Val)-Asp-fmk, Ac-Phg-Asp-fmk, Ac-(2-F-Phg)-Asp-fmk, Ac-(F3-Val)-Asp-fmk, Ac-(3-F-Val)-Asp-fmk, Z-Phg-Asp-fmk, Z-(2-F-Phg)-Asp-fmk, Z-(F3-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F3-Ala)-Asp-fmk, Z-(3-F-3-Me-Ala)-Asp-fmk, Z-(3-C1-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF3-Ala)-Asp-fmk, Z-(4-CF3-Phg)-Asp-fmk, Z-(3-Me2 N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, or Z-(2-Thg)-Asp-fmk, wherein Boc is tert-butylcarbonyl, Phg is phenylglycine, fmk is fluoromethylketone, Z is benzyloxycarbonyl, Chg is cyclohexlglycine, Fug is furylglycine, Thg is thienylglycine, Fua is furylalanine, Tha is thienylalanine, Nal is naphthylalanine, Cha is cyclohexlalanine, Abu is aminobutyric acid, Tle is tert-leucine, Cpg is cyclopentylglycine, Cbg is cyclobutylglycine and Thz is thioproline.

8. The method of claim 1, further comprising the step of contacting the kidney with one or more agents increase fibrocystin cleavage selected from modulators of microtubule polarization, modulators of Actin polymerization, modulators of contractile intracellular microfilaments and modulators of PKC activity.

9. The method of claim 8, wherein the agents are selected from Nocodazole, Benzolactam, Latrunculin, Cytochalasin B, Taxol and Bryostatin.

10. A composition for reducing cyst formation in a patient with polycystic kidney disease, the method comprising an effective amount of a fibrocystin cleavage inhibitor sufficient to reduce cyst formation in a patient, wherein the fibrocystin cleavage inhibitor comprises at least one of a proteasome inhibitor, a calpain inhibitor, and a β-secretase inhibitor.

11. The composition of claim 10, wherein the fibrocystin cleavage inhibitor is a proteasome inhibitor selected from the group consisting of: MG-132 (Carbobenzoxy-L-leucyl-L-leucyl-L-leucinal), MG-115 (Carbobenzoxy-L-leucyl-L-leucyl-L-norvalinal), PSI (Carbobenzoxy-L-isoleucyl-γ-t-butyl-L-glutamyl-L-alanyl-L-leucinal)-, Lactacystin (Synthetic: N-Acetyl-L-Cysteine, S-[2R,3 S,4R]-3-Hydroxy-2-[(1S)-1-Hydroxy-2-Methylpropyl-4-Methyl-5-Oxo-2-Pyrrolidinecarbonyl]), PS-519 (clasto-Lactacystin β-Lactone), α-Methylomuralide (α-Methyl clasto-Lactacystin β-Lactone), MG-101 (Ac-Leu-Leu-Nle-CHO), MG-262 (Z-Leu-Leu-Leu-B(OH)₂), PS-341 (Velcade; bortezomib), and Epoxomicin ((2R)-2-[Acetyl-(N-Methyl-L-Isoleucyl)-L-Isoleucyl-L-Threonyl-L-Leucyl]-2-Methyloxirane).

12. The composition of claim 10, wherein fibrocystin cleavage inhibitor is MG-132, MG-115, or MG-101 (calpain inhibitor 1).

13. The composition of claim 10, wherein the fibrocystin cleavage inhibitor is a calpain inhibitor selected from the group consisting of ritonavir, saquinavir, indinavir, nelfinavir, amprenavir.

14. The composition of claim 10, wherein fibrocystin cleavage inhibitor is a calpain inhibitor selected from Ac-Leu-Leu-Nle-H and Ac-Leu-Leu-Met-H.

15. The composition of claim 10, wherein fibrocystin cleavage inhibitor is a calpain inhibitor selected from Boc-Phg-Asp-fmk, Boc-(2-F-Phg)-Asp-fmk, Boc-(F3-Val)-Asp-fmk, Boc-(3-F-Val)-Asp-fmk, Ac-Phg-Asp-fmk, Ac-(2-F-Phg)-Asp-fmk, Ac-(F3-Val)-Asp-fmk, Ac-(3-F-Val)-Asp-fmk, Z-Phg-Asp-fmk, Z-(2-F-Phg)-Asp-fmk, Z-(F3-Val)-Asp-fmk, Z-Chg-Asp-fmk, Z-(2-Fug)-Asp-fmk, Z-(4-F-Phg)-Asp-fmk, Z-(4-Cl-Phg)-Asp-fmk, Z-(3-Thg)-Asp-fmk, Z-(2-Fua)-Asp-fmk, Z-(2-Tha)-Asp-fmk, Z-(3-Fua)-Asp-fmk, Z-(3-Tha)-Asp-fmk, Z-(3-Cl-Ala)-Asp-fmk, Z-(3-F-Ala)-Asp-fmk, Z-(F3-Ala)-Asp-fmk, Z-(3-F-3-Me-Ala)-Asp-fmk, Z-(3-C1-3-F-Ala)-Asp-fmk, Z-(2-Me-Val)-Asp-fmk, Z-(2-Me-Ala)-Asp-fmk, Z-(2-i-Pr-β-Ala)-Asp-fmk, Z-(3-Ph-β-Ala)-Asp-fmk, Z-(3-CN-Ala)-Asp-fmk, Z-(1-Nal)-Asp-fmk, Z-Cha-Asp-fmk, Z-(3-CF3-Ala)-Asp-fmk, Z-(4-CF3-Phg)-Asp-fmk, Z-(3-Me2 N-Ala)-Asp-fmk, Z-(2-Abu)-Asp-fmk, Z-Tle-Asp-fmk, Z-Cpg-Asp-fmk, Z-Cbg-Asp-fmk, Z-Thz-Asp-fmk, Z-(3-F-Val)-Asp-fmk, or Z-(2-Thg)-Asp-fmk, wherein Boc is tert-butylcarbonyl, Phg is phenylglycine, fink is fluoromethylketone, Z is benzyloxycarbonyl, Chg is cyclohexlglycine, Fug is furylglycine, Thg is thienylglycine, Fua is furylalanine, Tha is thienylalanine, Nal is naphthylalanine, Cha is cyclohexlalanine, Abu is aminobutyric acid, Tle is tert-leucine, Cpg is cyclopentylglycine, Cbg is cyclobutylglycine and Thz is thioproline.

16. The composition of claim 10, wherein the compositions further comprises one or more agents that cause fibrocystin cleavage selected from modulators of microtubule polarization, modulators of Actin polymerization, modulators of contractile intracellular microfilaments and modulators of PKC activity.

17. The composition of claim 16, wherein the agents are selected from Nocodazole, Benzolactam, Latrunculin, Cytochalasin B, Taxol and Bryostatin.

18. A method of reducing cyst formation in a patient with polycystic kidney disease, the method comprising:
contacting the kidney of the patient with an effective amount of a fibrocystin cleavage inducer sufficient to reduce cyst formation, wherein the fibrocystin cleavage inducer comprises at least one agent that increases an intracellular Ca⁺⁺ concentration wherein the levels of fibrocystin cleavage products increases.

19. The method of claim 18, wherein the agents are selected from thapsigargin, carbachol, caffeine and dantrolene.

20. A method of reducing cyst formation in a patient with polycystic kidney disease, the method comprising:
contacting the kidney of the patient with an effective amount of a fibrocystin cleavage inducer sufficient to reduce cyst formation, wherein the fibrocystin cleavage inducer comprises at least one agent that increases the activity of Protein Kinase C.

21. The method of claim 20, wherein the agents are selected from Benzolactam and Bryostatin.

22. The method of claim 20, further comprising at least one agent that increases an intracellular Ca⁺⁺ concentration wherein the levels of fibrocystin cleavage products increases.

23. A method of reducing cyst formation in a patient with polycystic kidney disease, the method comprising:
contacting the kidney with one or more agents that induce fibrocystin cleavage selected from modulators of microtubule polarization, modulators of Actin polymerization and modulators of intracellular microfilaments.

24. The method of claim 23, wherein the agents are selected from Nocodazole, Benzolactam, Latrunculin, Cytochalasin B, Taxol and Bryostatin.
